(19) United States
(12) Patent Application Publication
(10) Pub. No.: US 2020/0170732 A1
(43) Pub. Date: Jun. 4, 2020

(54) CONFIGURABLE PARALLEL MEDICAL ROBOT HAVING A COAXIAL END-EFFECTOR

(71) Applicant: KONINKLIJKE PHILIPS N.V., EINDHOVEN (NL)

(72) Inventor: Alexandru PATRICIU, BELMONT, MA (US)

(21) Appl. No.: 16/614,874

(22) PCT Filed: Jun. 19, 2018
(86) PCT No.: PCT/EP2018/066290

§ 371 Date: Nov. 19, 2019

Related U.S. Application Data
(60) Provisional application No. 62/521,618, filed on Jun. 19, 2017.

(51) Int. Cl. A61B 34/37; B25J 9/16; A61B 34/20; B25J 3/00; A61B 34/10; A61B 34/00; A61B 34/30

(52) U.S. Cl. CPC A61B 34/37; B25J 9/1664; A61B 34/20; A61B 2034/305; A61B 2034/107; A61B 34/25; A61B 2034/743; B25J 3/00

(57) ABSTRACT

A configurable parallel medical robot (30) employs a plurality of unassembled serial robot modules (40). Each serial robot module (40) includes a serial articulated robotic arm (50) and a serial end-effector (60). Each serial end-effector (60) includes a coaxial coupler (61), and the coaxial couplers are configured to coaxially couple two or more serial end-effectors (60) to form a coaxial end-effector (63) based on a plurality of configurations of the configurable parallel medical robot, each configuration including a different number of assembled serial robot modules (40). A parallel medical robotic system (20) employs a configuration controller (80) for determining a configuration of the configurable parallel medical robot (30) to robotically guide a medical tool (10) within a medical procedural space. The configuration controller (80) may further determine a mounting and/or a pose of the configuration of the parallel medical robot (30) within the medical procedural space.

### CROSS REFERENCE TO RELATED APPLICATIONS

This application is the U.S. National Phase application under 35 U.S.C. § 371 of International Application No. PCT/EP2018/066290 filed Jun. 19, 2018, published as WO 2018/234320 on Dec. 27, 2018, which claims the benefit of U.S. Provisional Patent Application No. 62/521,618 filed Jun. 19, 2017. These applications are hereby incorporated by reference herein.

### FIELD OF THE INVENTION

The present disclosure generally relates to medical robot systems for performing various medical procedures (e.g., laparoscopic surgery, neurosurgery, spinal surgery, natural orifice transluminal surgery, cardiology, pulmonary/bronchoscopy surgery, biopsy, ablation, and diagnostic interventions). The present disclosure specifically relates to a configurable parallel medical robot incorporating a coaxial connection of two or more serial end-effectors.

### BACKGROUND OF THE INVENTION

Medical robotics is a growing field that aims to improve the medical therapy delivery performance by robotic manipulators. Medical robots require accuracies comparable with industrial robots, but medical robots typically have lighter packages, lower speeds, and lower forces since medical robots work in the close proximity of a patient. Current medical robots have mainly serial architectures, which lead to bulky implementations due to the accuracy requirements. Further, a design of the serial robot manipulator is subject to conflicting requirements. On one hand, the manipulator shall have a high stiffness and accuracy, and on the other hand it should have a low mass. Low mass is a very desirable feature allowing for the medical robot to be attached to an interventional table mitigating potential issues that may arise due to a motion of the table with respect to a robot mount detached from the table.

More particularly, current medical robots typically are conventional serial structures and are mostly floor mounted. Some small medical robots with a parallel structure have been provided for specific applications. The main challenges that face current generation of medical robots may be summarized as follows.

First, current medical robots have bulky structures in order to achieve high accuracy and stiffness. This leads to floor attachments that is potentially hazardous because the table with the patient may move independently from the medical robot.

Second, the bulky structure of a medical robot may impede an imaging acquisition of the patient.

Finally, the bulky structure of a medical robot may impede access by a medical tool to the patient.

### SUMMARY OF THE INVENTION

The present disclosure describes a configurable parallel medical robot configured as a redundant parallel robot structure of serial robot modules based on two or more serial articulated robot arms that are coaxially coupled at serial end-effectors to form a coaxial end-effector. Each individual serial robot module is implemented with redundant degrees of freedom whereby an entirety of the parallel medical robot is capable of achieving a same positioning of the coaxial end-effector within a medical procedural space using many individual link poses of the serial articulated robot arms.

For purposes of describing and claiming the inventions of the present disclosure:

(1) the term “medical procedure” broadly encompasses all diagnostic, surgical and interventional procedures, as known in the art of the present disclosure or hereinafter conceived, for an imaging, a diagnosis and/or a treatment of a patient anatomy;

(2) the term “medical procedural space” broadly encompasses a coordinate space enclosing a medical procedure as exemplary described in the present disclosure;

(3) the term “medical tool” broadly encompasses, as understood in the art of the present disclosure and hereinafter conceived, a tool, an instrument, a device or the like for conducting an imaging, a diagnosis and/or a treatment of a patient anatomy. Examples of a medical tool include, but are not limited to, guidewires, catheters, scalpels, cauterizers, ablation devices, balloons, stents, endografts, atherectomy devices, clips, needles, forceps, k-wires and associated drivers, endoscopes, ultrasound probes, X-ray devices, awls, screwdrivers, osteotomes, chisels, mallets, curettes, clamps, forceps, periosteomes and j-needles;

(4) the term “serial articulated robotic arm” broadly encompasses all robotic arms, as known in the art of the present disclosure and hereinafter conceived, having an interconnected set of links and powered joints for supporting a translation, a rotation, and/or pivoting of an end-effector through a medical procedural space. Examples of serial articulated robotic arms include, but is not limited to, serial articulated robot arms employed by the da Vinci® Robotic System, the Medrobotics Flex® Robotic System, the Magellan™ Robotic System, and the CorePath® Robotic System;

(5) the term “serial end-effector” broadly encompasses all accessory devices, as known in the art of the present disclosure and hereinafter conceived, for attachment to a serial articular robotic arm for facilitating a performance of a task by the serial articulated robotic arm;

(6) the term “coaxial coupler” broadly encompasses any and all couplers, as known in the art of the present disclosure and hereinafter conceived, structurally configured to couple serial end effectors along a common radial axis as exemplary described in the present disclosure;

(7) the term “coaxial end-effector” broadly encompasses an end-effector formed by a coaxial coupling of the two or more serial end-effectors via the coaxial couplers as exemplary described in the present disclosure;

(8) the term “medical tool adapter” broadly encompasses any and all adapters, as known in the art of the present disclosure and hereinafter conceived, structurally configured to hold one or more types of medical tools as exemplary described in the present disclosure;

(9) the term “serial robot module” broadly encompasses a connected or a disconnected serial articulated robotic arm and serial end-effector pairing as known in the art of the present disclosure and hereinafter conceived;

(10) the term “configurable parallel medical robot” broadly encompasses an assembled or and an unassembled parallel configuration of two or more serial robot modules of the present disclosure for robotically guiding a medical tool within a medical procedural space as exemplary described in the present disclosure;

(11) the term “parallel medical robotic system” broadly encompasses all medical robotic systems incorporating a parallel medical robot of the present disclosure as exemplary described in the present disclosure;

(12) the term “medical imaging modality” broadly encompasses all imaging systems, as known in the art of the present disclosure and hereinafter conceived, for imaging a patient anatomy. Examples of an imaging system include, but is not limited to, a stand-alone x-ray imaging system, a mobile x-ray imaging system, an ultrasound imaging system (e.g., TEE, TTE, IVUS, ICE), computed tomography (“CT”) imaging system, positron emission tomography (“PET”) imaging system, and magnetic resonance imaging (“MRI”) system;

(13) the term “position tracking system” broadly encompasses all tracking systems, as known in the art of the present disclosure and hereinafter conceived, for tracking objects within a coordinate space. Examples of a robot tracking system include, but is not limited to, an electromagnetic (“EM”) tracking system (e.g., the Auora® electromagnetic tracking system), an optical-fiber based tracking system (e.g., Fiber-Optic RealShape™ (“FORS”) tracking system), an ultrasound tracking system (e.g., an InSitu or image-based US tracking system), an optical tracking system (e.g., a Polaris optical tracking system), a radio frequency identification tracking system and a magnetic tracking system;

(14) the term “controller” broadly encompasses all structural configurations, as understood in the art of the present disclosure and as exemplary described in the present disclosure, of an application specific main board or an application specific integrated circuit for controlling an application of various inventive principles of the present disclosure as subsequently described in the present disclosure. The structural configuration of the controller may include, but is not limited to, processor(s), computer-usable/computer readable storage medium(s), an operating system, application module(s), peripheral device controller(s), slot(s) and port(s). A controller may be housed within or linked to a workstation. Examples of a “workstation” include, but are not limited to, an assembly of one or more computing devices, a display/monitor, and one or more input devices (e.g., a keyboard, joysticks and mouse) in the form of a standalone computing system, a client computer of a server system, a desktop or a tablet;

(15) the descriptive labels for term “controller” herein facilitates a distinction between controllers as described and claimed herein without specifying or implying any additional limitation to the term “controller”;

(16) the term “application module” broadly encompasses an application incorporated within or accessible by a controller consisting of an electronic circuit and/or an executable program (e.g., executable software stored on non-transitory computer readable medium(s) and/or firmware) for executing a specific application;

(17) the terms “signal”, “data” and “command” broadly encompasses all forms of a detectable physical quantity or impulse (e.g., voltage, current, or magnetic field strength) as understood in the art of the present disclosure and as exemplary described in the present disclosure for transmitting information and/or instructions in support of applying various inventive principles of the present disclosure as subsequently described in the present disclosure. Signal/data/command communication between components of a coaxial medical robotic system of the present disclosure may involve any communication method as known in the art of the present disclosure including, but not limited to, signal/data/command transmission/reception over any type of wired or wireless datalink and a reading of signal/data/commands uploaded to a computer-usable/computer readable storage medium; and

(18) the descriptive labels for terms “signal”, “data” and “commands” herein facilitates a distinction between signals/data/commands as described and claimed herein without specifying or implying any additional limitation to the terms “signal”, “data” and “command”.

A first embodiment of the inventions of the present disclosure is a configuration parallel medical robot employing a plurality of serial robot modules. Each serial robot module includes a serial end-effector connected or connectable to a serial articulated robotic arm. Each serial end-effector includes a coaxial coupler for coaxially coupling two or more serial end-effectors to form a coaxial end-effector. One or more of the serial end-effectors may further include a medical tool adapter for holding a medical tool whereby a tool adapter may be integrated with or segregated from a corresponding coaxial coupler.

A second embodiment of the inventions of the present disclosure is a parallel medical robotic system employing a parallel medical robot of the first embodiment. The parallel medical robotic system employs a robot configuration controller for determining a configuration of the parallel medical robot to robotically guide a medical tool within a medical procedural space, the configuration including a coaxial coupling of two or more serial end-effector to form the coaxial end-effector. The robot configuration controller may further determine a mounting and/or a pose of the configuration of the parallel medical robot within the medical procedural space. The parallel medical robotic system may further employ a robot actuation controller for controlling an actuation of the configuration of the parallel medical robot within the medical procedural space.

A third embodiment of the inventions of the present disclosure is a method of operating a configurable parallel medical robot of the first embodiment. The method involves a robot configuration controller determining a configuration of the parallel medical robot to robotically guide a medical tool within a medical procedural space, the configuration including a coaxial coupling of two or more serial end-effectors to form the coaxial end-effector. The method further involves the robot configuration controller determining a mounting and/or a pose of the coaxial coupling of the configuration of the parallel medical robot within the medical procedural space.

The foregoing embodiments and other embodiments of the inventions of the present disclosure as well as various features and advantages of the present disclosure will become further apparent from the following detailed description of various embodiments of the inventions of the present disclosure read in conjunction with the accompanying drawings. The detailed description and drawings are merely illustrative of the inventions of the present disclosure rather than limiting, the scope of the inventions of present disclosure being defined by the appended claims and equivalents thereof.

### BRIEF DESCRIPTION OF THE DRAWINGS

FIG. 1 illustrates an exemplary embodiment of a parallel surgical robotic system in accordance with the inventive principles of the present disclosure.

FIGS. 2A and 2B illustrate an exemplary coaxial coupling of two (2) serial end-effectors in accordance with the inventive principles of the present disclosure.

FIGS. 3A and 3B illustrate an exemplary embodiment of a serial end-effector in accordance with the inventive principles of the present disclosure.

FIG. 4 illustrates an exemplary embodiment of a serial robot module in accordance with the inventive principles of the present disclosure.

FIG. 5A illustrates an exemplary embodiment of a parallel medical robot in accordance with the inventive principles of the present disclosure.

FIG. 5B illustrates an exemplary embodiment of a coaxial end-effector in accordance with the inventive principles of the present disclosure.

FIG. 6 illustrates an exemplary embodiment of a robot configuration controller in accordance with the inventive principles of the present disclosure.

FIG. 7 illustrates an exemplary medical procedure in accordance with the inventive principles of the present disclosure.

### DETAILED DESCRIPTION OF THE PREFERRED EMBODIMENTS

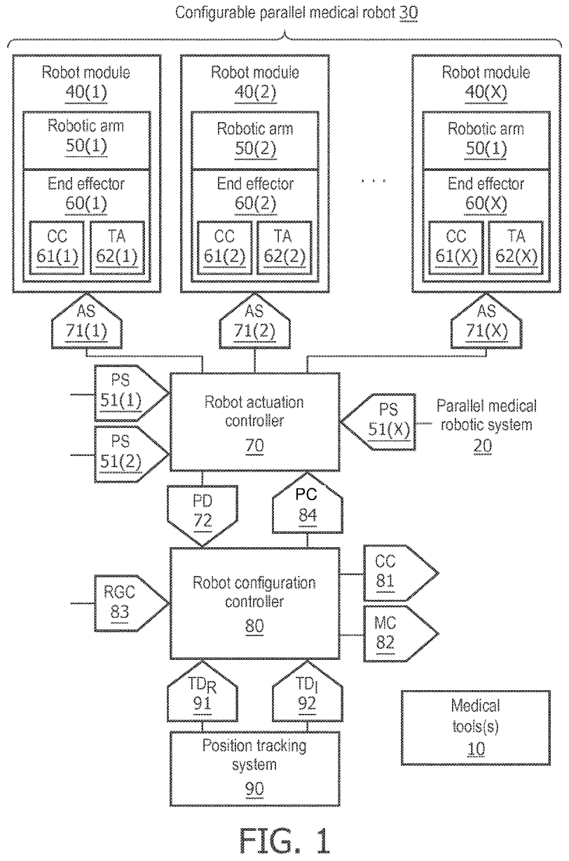

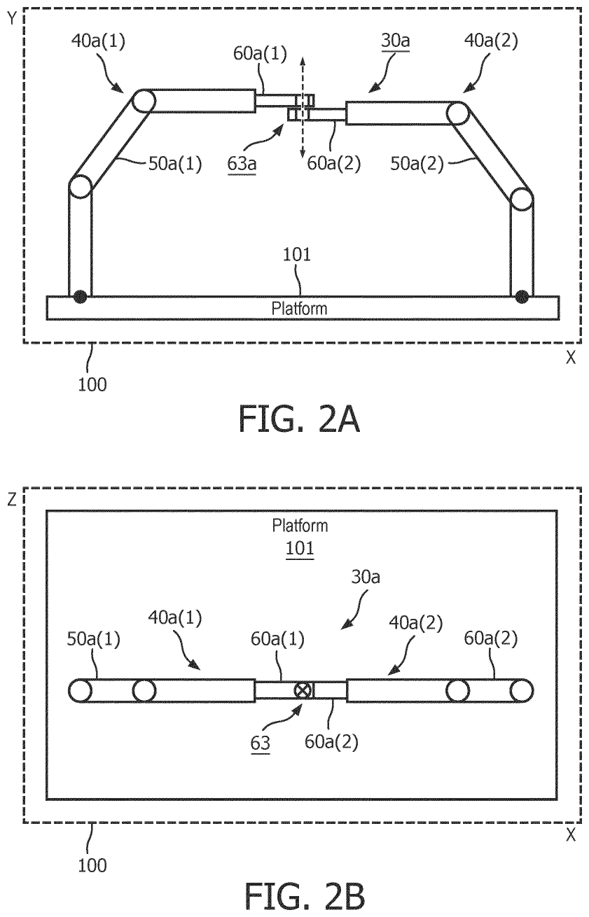

To facilitate an understanding of the inventions of the present disclosure, the following description of FIGS. 1-2B teaches basic inventive principles of an exemplary parallel medical robotic system 20 of the present disclosure. From this description, those having ordinary skill in the art will appreciate how to apply the inventive principles of the present disclosure to making and using numerous and varied embodiments of a parallel medical robotic system of the present disclosure.

Referring to FIG. 1, parallel medical robotic system 20 of the present disclosure provides robotic guidance for one or more medical tools 10 utilized to conduct an imaging, a diagnosis and/or a treatment of a patient anatomy in accordance with a medical procedure as known in the art of the present disclosure. Examples of a medical tool 10 include, but are not limited to, guidewires, catheters, scalpels, cauterizers, ablation devices, balloons, stents, endografts, atherectomy devices, clips, needles, forceps, k-wires and associated drivers, endoscopes, ultrasound probes, X-ray devices, awls, screwdrivers, osteotomes, chisels, mallets, curettes, clamps, forceps, periosteomes and j-needles.

In practice, the robotic guidance of a medical tool 10 is dependent upon the particular medical procedure. Examples of such robotic guidance include, but are not limited to, an image based robotic guidance and a master slave type of robotic guidance.

Still referring to FIG. 1, parallel medical robotic system 20 employs a configurable parallel medical robot 30, a robot actuation controller 70 and a robot configuration controller 80, and may further employ a position tracking system 90.

Configurable parallel medical robot 30 includes a X number of serial robot modules 40, X≥1, whereby a Y number of serial robot modules 40 are selected by robot configuration controller 80 for configuring parallel medical robot 30 to robotically guide a medical tool within a medical procedural space (e.g., an operating room, a training room, etc.) as will be further described in the present disclosure.

Each serial robot module 40 includes a serial articulated robotic arm 50 as known in the art of the present disclosure and a serial end effector 60 in accordance with the inventive principles of the present disclosure.

In practice, serial robot modules 40 may be identical or functional equivalent whereby redundancy is introduced into system 20.

Serial articulated robotic arm 50 employs a linkages (not shown) including a proximal linkage, a distal linkage and optionally including one or more intermediate linkages. Serial articulated robotic arm 50 further includes actuator joint(s) (not shown) interconnecting the linkages in a complete or partial serial arrangement. Each actuator joint is actuatable by robot actuation controller 70 via actuation signals 71 for controlling a pose of each linkage as known in the art of the present disclosure, and each actuator joint includes a pose sensor of any type (e.g., an encoder) for generating a pose signal 51 informative of a pose (i.e., orientation and/or location) of each linkage relative to a reference as known in the art of the present disclosure.

In practice, an actuator joint may be of any type of actuator joint as known in the art including, but not limited to, a translational actuator joint, a ball and socket actuator joint, a hinge actuator joint, a condyloid actuator joint, a saddle actuator joint and a rotary actuator joint.

Also in practice, each serial articulated robotic arm 50 may be the same type of serial articulated robotic arm, different types of serial articulated robotic arm, or a mixture of same and different types of serial articulated robotic arm.

Serial end-effector 60 includes an end-effector utilized by serial articulated robotic arms as known in the art of the present disclosure that incorporates a coaxial coupler 61 and optionally a tool adapter 62 as will be further described in the present disclosure.

In practice, each serial end-effector 60 may be the same type of end-effector, different types of end-effectors, or a mixture of same and different types of end-effectors.

Also in practice, each serial end-effector 60 may have the same geometrical shape and same dimensions, different geometrical shapes and same dimensions, or different geometrical shapes and different dimensions.

The present disclosure provides for a coaxial coupling of two or more serial end-effectors 60 via coaxial couplers 61 to form a coaxial end-effector to support a performance of the medical procedure within a medical procedural space.

For example, FIGS. 2A and 2B illustrate a configuration 30a of a configurable parallel medical robot 30 mounted on a platform 101 within a medical procedural space 100 to support a performance of a medical procedure within medical procedural space 100. Parallel medical robot 30a has two (2) serial robot modules 40a whereby the serial end-effector 60a are coaxially coupled along a common radial axis represented by the dashed bi-directional arrow. The coaxial coupling of serial end-effectors 60a forms a coaxial end-effector 63 that may be translated, rotated and/or pivoted to a desired position within medical procedural space 100 by controllable actuations of serial articulated robotic arms 50 as known in the art of the present disclosure and further described in the present disclosure.

Referring back to FIG. 1, robot configuration controller 80 performs three (3) main tasks of the present disclosure.

First, robot configuration controller 80 preoperatively determines a number Y of robot modules for configuring parallel medical robot 30 in support of the medical procedure as will be further described in the present disclosure, Y≤X. Robot configuration controller 80 communicates capacity configuration data 81 informative of the number of Y robot modules to appropriate personnel, such as, for example, by a display and/or a printing of capacity configuration data 81, whereby the personnel may identify the number of Y robot modules needed for robotically guiding the medical tool 10 within the medical procedural space.

In practice, for embodiments of configurable parallel medical robot 30 having different types of serial articulate robotic arms 50 and/or different types of serial end-effectors 60, capacity configuration data 81 may further be informative of which types of serial articulate robotic arms 50 and/or which types of serial end-effectors 60 are to be utilized for robotically guiding the medical tool 10 within the medical procedural space.

Second, robot configuration controller 80 preoperatively determines a mounting of the Y number of serial robot modules 40 within the medical procedural space suitable in support of the medical procedure as will be further described in the present disclosure. Robot configuration controller 80 communicates mounting configuration data 82 informative of a mounting of the number of Y of robot modules 40 to appropriate personnel, such as, for example, by a display and/or a printing of mounting configuration data 82, whereby the personnel may identify the mounting location of each robot module 40 within the medical procedural space.

Third, robot configuration controller 80 intraoperatively processes robot guidance commands 83 informative of a desired robotic guidance of the medical tool within the medical procedural space as known in the art of the present disclosure (e.g., image guided commands, user input commands, etc.) to thereby intraoperatively determine poses of each robot module 40 for robotically guiding the medical tool 10 in accordance with the robot guidance commands 83.

Additionally, robot actuation controller 70 intraoperatively generates pose data 72 informative of a current pose (i.e., orientation and/or location) of each linkage relative to a reference as known in the art of the present disclosure. Robot configuration controller 80 intraoperatively processes pose data 72 to thereby generate pose commands 84 informative of the determined poses of each robot module 40 in accordance with the robot guidance commands 83 whereby robot actuation controller 70 generates actuation signals 71 in accordance with the pose commands 84.

In practice, robot configuration controller 80 may generate pose commands 84 based on additional criteria, such as, for example, a stiffness optimization, exclusion zones and/or imager interference minimization as will be further described in the present disclosure.

Also in practice, if position tracking system 90 is employed, robot configuration controller 80 may generate pose commands 84 based on a generation by position tracking system 90 of robot tracking data 91 informative of a tracked position of the coaxial end-effector within the medical procedural space as known in the art of the present disclosure and/or based a generation by position tracking system 90 of imager tracking data 92 informative of a tracked position of a medical imaging modality (not shown) (e.g., an X-ray/CT modality) within the medical procedural space as known in the art of the present disclosure.

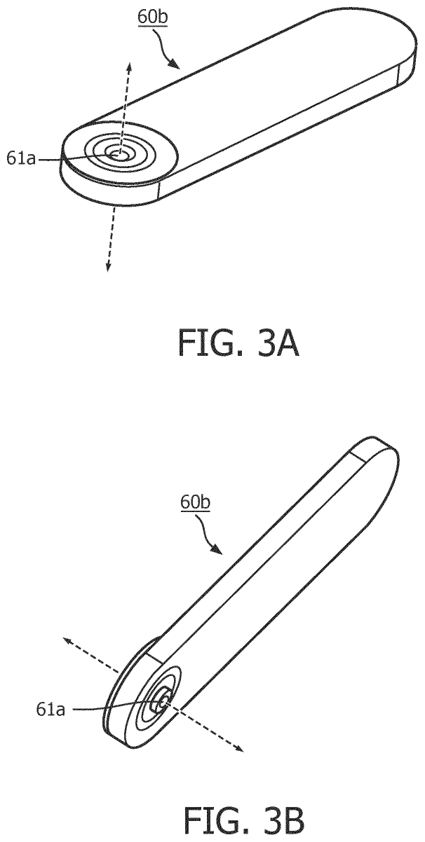

To facilitate a further understanding of the inventions of the present disclosure, the following description of FIGS. 3A-5 teaches basic inventive principles of an exemplary configuration 30b of a configurable parallel medical robot 30 (FIG. 1) of the present disclosure. From this description, those having ordinary skill in the art will appreciate how to apply the inventive principles of the present disclosure to making and using numerous and varied configuration embodiments of a configurable parallel medical robot of the present disclosure.

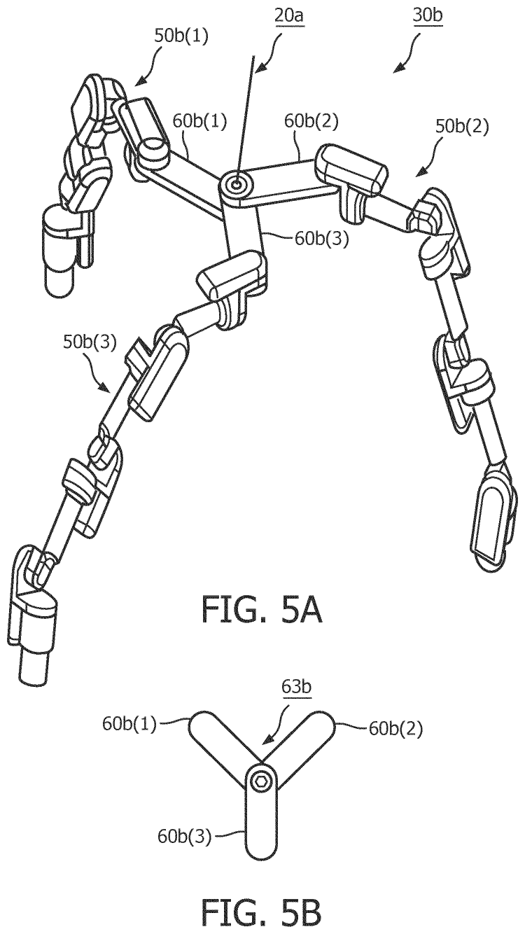

The parallel medical robot 30b as shown in FIG. 5 employs three robot module, each consisting of a serial articulated robotic arm 50b and a serial end-effector 60b.

Referring to FIGS. 3A and 3B, serial end-effector 60b in the form of an elongated disk includes a coaxial coupler 61a on a distal end of serial end-effector 60b. Coaxial coupler 61b has a radial axis symbolized by a dashed bi-directional arrow whereby two or more coaxial couplers 61a may be coaxially coupled along the radial axes. Integrated within coaxial coupler 61a is a medical tool adapter (not shown) for holding a medical tool 10 (FIG. 1). In practice, coaxial coupler 61a may incorporate a bearing centered on the radial axis if a rotation of the medical tool 10 is not required.

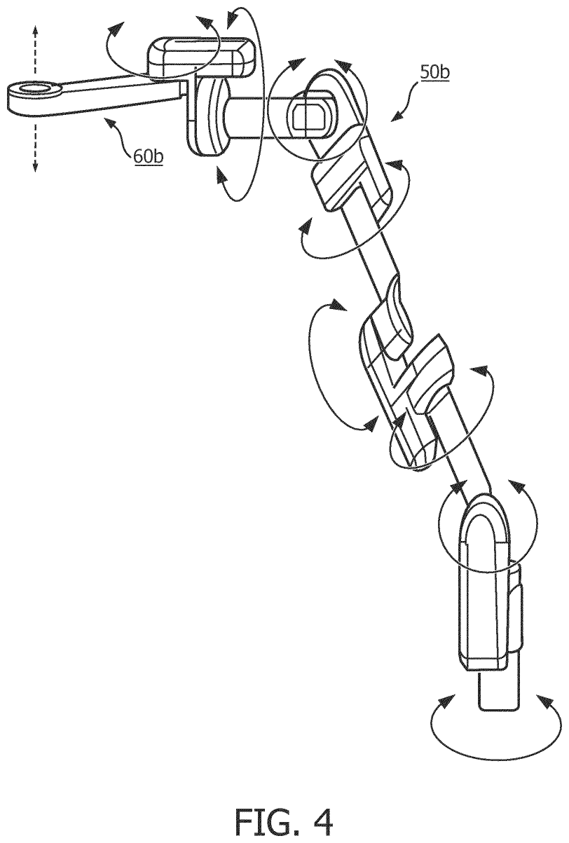

Referring to FIG. 4, a serial articulated robotic arm 50b includes a series of links and active joints with orthogonal rotation axes as known in the art of the present disclosure. A proximal end of serial end-effector 60b is permanently affixed or detachable coupled to a distal link of serial articulated robotic arm 50b.

Referring to FIG. 5A, the serial end-effector 60b are coaxially coupled to form a coaxial end-effector 63b as shown in FIG. 5B. Coaxial end-effector 63b is holding a needle 20a having a distal tip calibrated within coaxial end-effector 63b whereby active control of the joints of each serial articulated robotic arm 50b facilitates a translation, a pivoting and/or a rotation of coaxial end-effector 63b via poses of arms 50b to position the distal tip of needle 20 to desired position within a medical procedural space. Of importance is the redundancy of parallel medical robot 30b whereby parallel medical robot 30a is operable via actuation signals to achieve a same calibrated position of coaxial end-effector 63b using many different poses of serial articulated robotic arms 50b as would be appreciated those skilled in the art of the present disclosure.

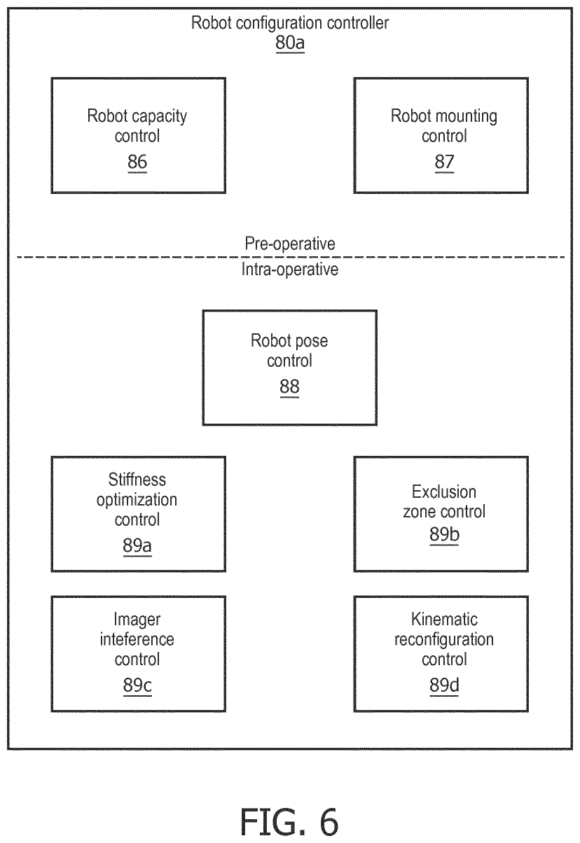

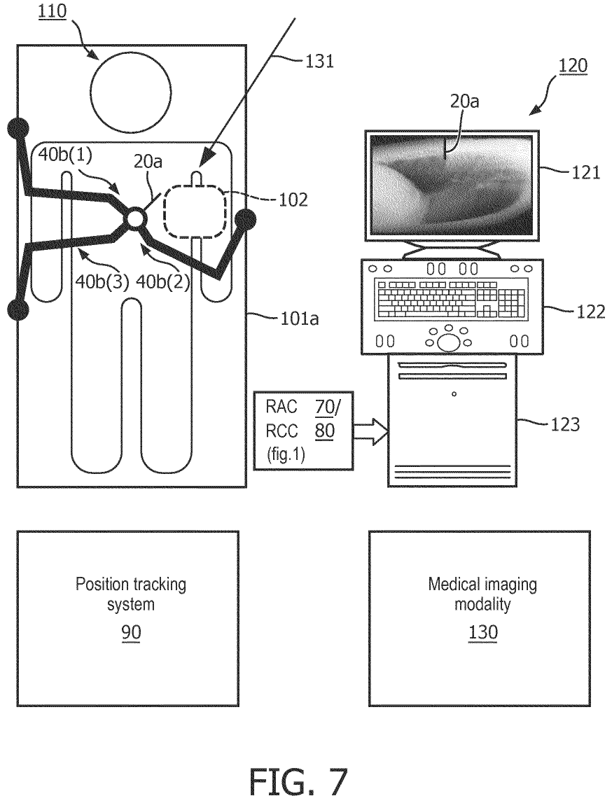

To facilitate a further understanding of the inventions of the present disclosure, the following description of FIGS. 6 and 7 teaches basic inventive principles of an exemplary embodiment 80a of robot configuration controller 80 (FIG. 1) of the present disclosure. From this description, those having ordinary skill in the art will appreciate how to apply the inventive principles of the present disclosure to making and using numerous and varied embodiments of a robot configuration controller of the present disclosure.

Referring to FIG. 6, robot configuration controller 80a includes a set of preoperative modules for initially configuring and mounting a configurable parallel medical robot 30 (FIG. 2), and a set of intraoperative modules for posing the redundant parallel medical robot 30 in view of various criteria.

The set of preoperative modules includes a robot capacity control module 86 to preoperatively determines a number Y of robot modules 40 for configuring parallel medical robot 30 in support of the medical procedure, Y≤X.

In practice, robot capacity control module 86 may implement any technique known in the art of the present disclosure and described within the present disclosure for determining a number Y of serial robot modules 40 for configuring parallel medical robot 30 in support of the medical procedure.

In one embodiment, robot capacity control module 86 computes a required number of serial robot modules 40 by dividing a load by a medical tool 12 by a load capacity of each serial robot module 40 and rounding up the quotient to the next integer.

For example, robot capacity control module 86 may compute three (3) serial robot modules 40b for handling a load of a needle 20 as shown in FIG. 7.

The set of preoperative modules further includes a robot mounting control module 87 to preoperatively determine a mounting of the Y number of serial robot modules 40 within the medical procedural space suitable in support of the medical procedure.

In practice, robot mounting control module 87 may implement any technique known in the art of the present disclosure and described within the present disclosure for determining a mounting of the Y number of serial robot modules 40 within the medical procedural space suitable in support of the medical procedure.

In one embodiment, robot mounting control module 87 is designed to consider an optimization of mounting positions of serial robot modules 40 on a patient table optimization problem. Specifically, n joint variables of each serial robot module i is labeled θᵢₗ . . . θᵢₙ. A position of a table attachment a of the ith serial robot module is aᵢ. The position of the ith serial end-effector in a global coordinate system is K(aᵢ, θᵢₗ . . . θᵢₙ). The stiffness of the ith serial robot module is S(aᵢ, θᵢₗ . . . θᵢₙ). Generally, the stiffness is a 6×6 matrix that relates the deflection of the serial end-effector to the loads applied to the serial end-effector. The goal for the optimization is to find the aᵢ for i=1 . . . n such that all the serial robot modules can reach the desired position and the stiffness is maximized. The stiffness of the entire structure is the sum of individual module stiffness. The stiffness may be optimized with respect to all directions or with respect to certain direction.

To optimize the positioning such that the global stiffness is maximized, then find aᵢ, . . . , aᵣ such that Minimize ((1−ConditionNumber(S))\hat{ }2+(1/Smallest EigenValue(S))\hat{ }2) such that K_{d}=K(aₗθₗₗ . . . θₗₙ)=K(a₂, θ₂ₗ . . . θ₂ₙ=K(aᵢ, θᵢₗ . . . θᵢₙ) for any K_d in the desired workspace.

This is a constrained optimization problem whereby the ConditionNumber of a matrix is the ratio between the minimum and maximum eigenvalues of the matrix. The first term that has to be minimized requires that the eigenvalues are clustered together, i.e., that is the stiffness has the same characteristics in all directions. The second term maximizes the smallest eigenvalue that is it maximizes the global stiffness. The problem can be solved using classical optimization solvers as known in the art of the present disclosure.

For example, robot mounting control module 87 may determine a mounting of the three (3) serial robot modules 40b on a patient table 101a as shown in FIG. 7 in view of optimizing a stiffness of serial robot modules 40b, which optimizes an overall stiffness of parallel medical robot 30b.

In practice, robot mounting control module 87 may consider other criteria including, but not limited to, a particular directions stiffness and/or defining zones in which the robot links should not enter.

The set of intraoperatively modules includes a robot pose control module 88 to intraoperatively determine poses of each serial robot module 40 for robotically guiding the medical tool 10 in accordance with the robot guidance commands and optionally tracking data previously described in the present disclosure.

In practice, robot pose control module 88 may implement any technique for identifying each pose of redundant serial robot modules 40 for robotically guiding medical tool 10 to a desired position within the medical procedural space and for selecting one of the identified poses of redundant serial robot modules 40 to thereby robotically guide medical tool 10 to the desired position within the medical procedural space as known in the art of the present disclosure and exemplary described in the present disclosure.

In one embodiment, robot pose control module 88 may implement a backlash elimination scheme by selecting a pose providing antagonistic action between serial robot modules 40.

In another embodiment, in view of a position of the coaxial end-effector being provided at any given time by several kinematic chains, robot pose control module 88 processes pose data to determine if any actuator has failed to thereby select a pose supported by the remaining enabled actuators.

The set of intraoperatively modules further includes a stiffness optimization control module 89a to supplement robot pose control module 88 for pose optimization. Specifically, stiffness optimization control module 89a identifies θₗₗ . . . θᵣₙ that Minimize CF(θₗₗ . . . θᵣₙ) subject to constraints Kd=K(aₗ, θₗₗ . . . θₗₙ)=K(a₂, θ₂ₗ . . . θ₂ₙ)= . . . =K(aᵢ, θᵢₗ . . . θᵢₙ), where Kd is the desired position of the coaxial end-effector computed from a desired target position and CF is a cost function that implements the desired robot behaviour. Several CF functions can be defined including but not limited to CF(θₗₗ . . . θᵣₙ)=1/∥ S(θₗₗ . . . θᵣₙ)∥, where ∥·∥ is the matrix norm.

For example, robot pose control module 88 provide a stiffness optimization of serial robot modules 40b as shown in FIG. 7 based on a desired target position of the calibrated coaxial end-effector.

The set of intraoperatively modules further includes an exclusion zone control module 89b to supplement robot pose control module 88 for pose optimization. Specifically, exclusion zone control module 89b delineates any exclusion zone of the medical procedural space bounded by set of planes with equations pⱼ(Pt)=0, j=1 . . . m, where p is the equation that defines the plane and Pt a point in the 3D space. Acceptable subspace is defined as the set of points Pt such that p_{J} (Pt)<0. Exclusion zone control module 89b ensures some or all robot links and joints are within the acceptable subspace of the medical procedural space by satisfying a constraint pⱼ(Pt)>=0 for any Pt that belongs to robot geometry and any j=1 . . . m.

For example, exclusion zone control module 89b monitors robot modules 40b to ensure none of the links or joints of a serial robot module 40b are within an exclusion zone 102 delineated as an surgeon position providing access to patient 110.

The set of intraoperatively modules further includes an imager interference control module 89c to supplement robot pose control module 88 for pose optimization. Specifically, based on a tracked position of a parallel medical robot relative to tracked position of a medical imaging modality, imager interference control module 89c delineates any point of the parallel medical robot within an imaging volume of the medical imaging modality.

For examples, referring to FIG. 7, position tracking system 90 tracks a position of serial robot modules 40 (e.g., sensors attached to serial end-effector) within the medical procedural space and a position of a field of view 131 of a medical imaging modality 130 within the medical procedural space whereby imager interference control module 89c delineates any point of serial robot modules 40 within an imaging volume of the medical imaging modality 130.

The set of intraoperatively modules further includes a kinematic reconfiguration control module 89d to supplement robot pose control module 88 for pose optimization. Specifically, kinematic reconfiguration control module 89d may implement a virtual Remote Centre of Motion or other specific kinematic structures by making active joints passive or using electronic gearing between joints.

In practice, robot pose control module 88 may temporally choose between one or more criteria for pose optimization. For example, as shown in FIG. 7, robot pose control module 88 may optimize a stiffness of serial robot modules 40b during an insertion of a medical tool 20 into patient 110 while robot pose control module 88 may minimize imager interface during an imaging sequence of the insertion of medical tool 20 into patient 110.

Referring to FIG. 7, robot actuation controller 70 (FIG. 1) and robot configuration controller 80 (FIG. 1) are installed on a workstation 120 including a known arrangement of a monitor 121, a keyboard 122 and a computer 123 as known in the art of the present disclosure.

As installed, robot actuation controller 70 and robot configuration controller 80 each may include a processor, a memory, a user interface, a network interface, and a storage interconnected via one or more system buses.

The processor may be any hardware device, as known in the art of the present disclosure or hereinafter conceived, capable of executing instructions stored in memory or storage or otherwise processing data. In a non-limiting example, the processor may include a microprocessor, field programmable gate array (FPGA), application-specific integrated circuit (ASIC), or other similar devices.

The memory may include various memories, as known in the art of the present disclosure or hereinafter conceived, including, but not limited to, L1, L2, or L3 cache or system memory. In a non-limiting example, the memory may include static random access memory (SRAM), dynamic RAM (DRAM), flash memory, read only memory (ROM), or other similar memory devices.

The user interface may include one or more devices, as known in the art of the present disclosure or hereinafter conceived, for enabling communication with a user such as an administrator. In a non-limiting example, the user interface may include a command line interface or graphical user interface that may be presented to a remote terminal via the network interface.

The network interface may include one or more devices, as known in the art of the present disclosure or hereinafter conceived, for enabling communication with other hardware devices. In an non-limiting example, the network interface may include a network interface card (NIC) configured to communicate according to the Ethernet protocol. Additionally, the network interface may implement a TCP/IP stack for communication according to the TCP/IP protocols. Various alternative or additional hardware or configurations for the network interface will be apparent\

The storage may include one or more machine-readable storage media, as known in the art of the present disclosure or hereinafter conceived, including, but not limited to, read-only memory (ROM), random-access memory (RAM), magnetic disk storage media, optical storage media, flash-memory devices, or similar storage media. In various non-limiting embodiments, the storage may store instructions for execution by the processor or data upon with the processor may operate. For example, the storage may store a base operating system for controlling various basic operations of the hardware. The storage may further store one or more application modules in the form of executable software/firmware.

Still referring to FIG. 7, in practice, robot actuation controller 70 and robot configuration controller 80 may be partially or wholly integrated within workstation 120. Also in practice, robot actuation controller 70 and robot configuration controller 80 may be installed on different workstations and operate via a wired/wireless communication scheme as known in art of the present disclosure.

Referring to FIGS. 1-7, those having ordinary skill in the art of the present disclosure will appreciate numerous benefits of the inventions of the present disclosure including, but not limited to, an optimization of a parallel medical robotic structure.

Furthermore, as one having ordinary skill in the art will appreciate in view of the teachings provided herein, features, elements, components, etc. described in the present disclosure/specification and/or depicted in the Figures may be implemented in various combinations of electronic components/circuitry, hardware, executable software and executable firmware and provide functions which may be combined in a single element or multiple elements. For example, the functions of the various features, elements, components, etc. shown/illustrated/depicted in the Figures can be provided through the use of dedicated hardware as well as hardware capable of executing software in association with appropriate software. When provided by a processor, the functions can be provided by a single dedicated processor, by a single shared processor, or by a plurality of individual processors, some of which can be shared and/or multiplexed. Moreover, explicit use of the term “processor” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, circuitry, combinations thereof, etc.) which is capable of (and/or configurable) to perform and/or control a process.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar function, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Furthermore, exemplary embodiments of the present disclosure can take the form of a computer program product or application module accessible from a computer-usable and/or computer-readable storage medium providing program code and/or instructions for use by or in connection with, e.g., a computer or any instruction execution system. In accordance with the present disclosure, a computer-usable or computer readable storage medium can be any apparatus that can, e.g., include, store, communicate, propagate or transport the program for use by or in connection with the instruction execution system, apparatus or device. Such exemplary medium can be, e.g., an electronic, magnetic, optical, electromagnetic, infrared or semiconductor system (or apparatus or device) or a propagation medium. Examples of a computer-readable medium include, e.g., a semiconductor or solid state memory, magnetic tape, a removable computer diskette, a random access memory (RAM), a read-only memory (ROM), flash (drive), a rigid magnetic disk and an optical disk. Current examples of optical disks include compact disk—read only memory (CD-ROM), compact disk—read/write (CD-R/W) and DVD. Further, it should be understood that any new computer-readable medium which may hereafter be developed should also be considered as computer-readable medium as may be used or referred to in accordance with exemplary embodiments of the present disclosure and disclosure.

Having described preferred and exemplary embodiments of novel and inventive coaxial medical robots and coaxial medical robotic systems (which embodiments are intended to be illustrative and not limiting), it is noted that modifications and variations can be made by persons having ordinary skill in the art in light of the teachings provided herein, including the Figures. It is therefore to be understood that changes can be made in/to the preferred and exemplary embodiments of the present disclosure which are within the scope of the embodiments disclosed herein.

Moreover, it is contemplated that corresponding and/or related systems incorporating and/or implementing the device or such as may be used/implemented in a device in accordance with the present disclosure are also contemplated and considered to be within the scope of the present disclosure. Further, corresponding and/or related method for manufacturing and/or using a device and/or system in accordance with the present disclosure are also contemplated and considered to be within the scope of the present disclosure.

1. A parallel medical robotic system, comprising:
a configurable parallel medical robot including a plurality of unassembled serial robot modules,
wherein each serial robot module includes a serial articulated robotic arm and a serial end-effector,
wherein each serial end-effector includes a coaxial coupler; and—
wherein the coaxial couplers are configured to coaxially couple at least two serial end-effectors to form a coaxial end-effector based on a plurality of configurations of the configurable parallel medical robot, each configuration including a different number of assembled serial robot modules.

2. The parallel medical robotic system of claim 1,
wherein at least one serial end-effector includes a medical tool adapter configured to hold a medical tool.

3. The parallel medical robotic system of claim 1,
wherein at least one coaxial coupler includes a medical tool adapter configured to hold a medical tool.

4. The parallel medical robotic system of claim 1, further comprising:
a robot actuation controller configured to control an actuation of the configurable parallel medical robot to robotically guide a medical tool within a medical procedural space.

5. The parallel medical robotic system of claim 1, further comprising:
a robot configuration controller configured to control a determination of a configuration of the configurable parallel medical robot to robotically guide a medical tool within a medical procedural space.

6. The parallel medical robotic system of claim 5,
wherein the robot configuration controller is further configured to determine a number of at least two serial robot modules for configuring the configurable parallel medical robot in the determined configuration based on a load of a medical tool and on a medical tool load capacity of each serial robot module.

7. The parallel medical robotic system of claim 1, further comprising:
a robot configuration controller configured to control a determination of a mounting of a configuration of the configurable parallel medical robot within a medical task space to robotically guide a medical tool within the medical task space.

8. The parallel medical robotic system of claim 7,
wherein the robot configuration controller is configured to determine the mounting of the configuration of the configurable parallel medical robot within the medical task space based on a stiffness of the configuration of the configurable parallel medical robot.

9. The parallel medical robotic system of claim 1, further comprising:
a robot configuration controller configured to control a determination of a pose of a configuration of the configurable parallel medical robot to robotically guide a medical tool within a medical task space.

10. The parallel medical robotic system of claim 9,
wherein the robot configuration controller is configured to determine the pose of the configuration of the configurable parallel medical robot within the medical task space based on a desired position of the coaxial end-effector within the medical procedural space.

11. The parallel medical robotic system of claim 9,
wherein the robot configuration controller is configured to determine the pose of the configuration of the configurable parallel medical robot within the medical task space based on a stiffness of the configuration of the parallel medical robot.

12. The parallel medical robotic system of claim 9,
wherein the robot configuration controller is configured to determine the pose of the configuration of the configurable parallel medical robot within the medical task space based on at least one exclusion zone within the medical procedural space.

13. The parallel medical robotic system of claim 9,
wherein the robot configuration controller is configured to determine the pose of the configuration of the configurable parallel medical robot within the medical task space based on a tracked position of the coaxial end-effector within the medical procedural space relative to a tracked position of a medical imaging modality within the medical procedural space.

14. The parallel medical robotic system of claim 9,
wherein the robot configuration controller is configured to determine the pose of the configuration of the configurable parallel medical robot within the medical task space based on any actuation failure of the configuration of the configurable parallel medical robot within the medical procedural space.

15. The parallel medical robotic system of claim 1, further comprising:
a robot configuration controller configured to control an active kinematic configuration of the configurable parallel medical robot within a medical procedural space.

16. A method of operating a configurable parallel medical robot including a plurality of serial robot modules, wherein each serial robot module includes a serial articulated robotic arm and a serial end-effector, wherein each serial end-effector includes a coaxial coupler configured to coaxially couple at least two serial end-effector to form a coaxial end-effector,
the method comprising:
a robot configuration controller determining a configuration of the parallel medical robot to robotically guide a medical tool within a medical procedural space,
wherein the configuration of the parallel medical robot includes a coaxial coupling of at least two serial end-effector to form the coaxial end-effector; and
the robot configuration controller determining a mounting of the configuration of the parallel medical robot within the medical procedural space.

17. The method of claim 16,
wherein the robot configuration controller determines the mounting of the configuration of the parallel medical robot within the medical procedural space based on a stiffness of the configuration of the parallel medical robot.

18. The method of claim 16, further comprising:
a robot configuration controller determining a pose of the configuration of the parallel medical robot within the medical procedural space.

19. The method of claim 18, further comprising:
wherein the robot configuration controller determines the pose of the configuration of the parallel medical robot within the medical procedural space based on at least one of:
a desired positioning of the coaxial end-effector within the medical procedural space;
a stiffness of the configuration of the parallel medical robot;
at least one exclusion zone within the medical procedural space;
a tracked position of the coaxial end-effector within the medical procedural space relative to a tracked position of a medical imaging modality within the medical procedural space; and
any actuation failure of the configuration of the parallel medical robot within the medical procedural space.

20. The method of claim 16, further comprising:
the robot configuration controller controlling an active kinematic configuration of the parallel medical robot within the medical procedural space.
